(19) United States
(12) Patent Application Publication
(10) Pub. No.: US 2017/0332914 A1
(43) Pub. Date: Nov. 23, 2017

(54) NON-INVASIVE BODY MONITOR

(71) Applicant: Daylight Solutions, Inc., San Diego, CA (US)

(72) Inventors: William B. Chapman, San Diego, CA (US); Miles James Weida, Poway, CA (US)

(73) Assignee: Daylight Solutions, Inc., San Diego, CA (US)

(21) Appl. No.: 15/524,541

(22) PCT Filed: Nov. 12, 2015
(86) PCT No.: PCT/US2015/060459

§ 371 Date: May 4, 2017

Related U.S. Application Data
(60) Provisional application No. 62/079,249, filed on Nov. 13, 2014.

(51) Int. Cl. A61B 5/00; A61B 5/145

(52) U.S. Cl. CPC A61B 5/0095; A61B 5/681; A61B 5/1451; A61B 5/14532

(57) ABSTRACT

A body monitor (12) for monitoring a condition of a living being (10) includes (i) a monitor housing (28) that is positioned adjacent to the living being (10); (ii) a first laser source (240) that directs a first output beam (240A) at the living being (10) to generate first photoacoustic waves; (iii) a second laser source (242) that directs a second output beam (242A) at the living being (10) to generate second photoacoustic waves; and (iv) a photoacoustic detector (16) secured to the monitor housing (28). The photoacoustic detector (16) detects the first photoacoustic waves and the second photoacoustic waves to monitor the condition of the living being (10). The output beams (240A) (240B) have a different center wavelength and can be in the mid-infrared range.

### RELATED APPLICATION

This application claims priority on U.S. Provisional Application Ser. No. 62/079,249, filed Nov. 13, 2014 and entitled “NON-INVASIVE BODY MONITOR”. As far as permitted, the contents of U.S. Provisional Application Ser. No. 62/079,249 are incorporated herein by reference.

### BACKGROUND

Blood glucose monitoring is a way to test the concentration of glucose in the blood. For example, blood glucose monitoring is an important part of the care of diabetes. One common method to monitor the concentration of glucose is to pierce the skin, draw blood, and apply the blood to a chemically active test strip.

### SUMMARY

The present invention is directed to a non-invasive body monitor for monitoring a condition of a living being. The body monitor can include (i) a monitor housing having a body side that is adapted to be positioned adjacent to and against the living being; (ii) a first laser source that directs a first output beam at the living being to generate first photoacoustic waves, the first output beam having a first center wavelength in the mid-infrared range, the first laser source being secured to the monitor housing adjacent to the body side; (iii) a second laser source that directs a second output beam at the living being to generate second photoacoustic waves, the second output beam having a second center wavelength in the mid-infrared range, the second center wavelength being different from the first center wavelength, the second laser source being secured to the monitor housing adjacent to the body side; and (iv) a photoacoustic detector secured to the monitor housing adjacent to the body side, the photoacoustic detector detecting the first photoacoustic waves and the second photoacoustic waves in order to monitor the condition of the living being. For example, the body monitor can be used to monitor a glucose level in the living being.

In one embodiment, each laser source includes a surface emitting, ring shaped quantum cascade laser, and the photoacoustic detector is a micro ring resonator assembly. For example, the body monitor can be used to monitor a glucose level in the living being.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified side view of a portion of a living being and a body monitor having one or more features of the present invention;

FIG. 2 is a simplified side view of the body monitor of FIG. 1; and

FIG. 3 is a simplified view of a photoacoustic detector having features of the present invention.

### DESCRIPTION

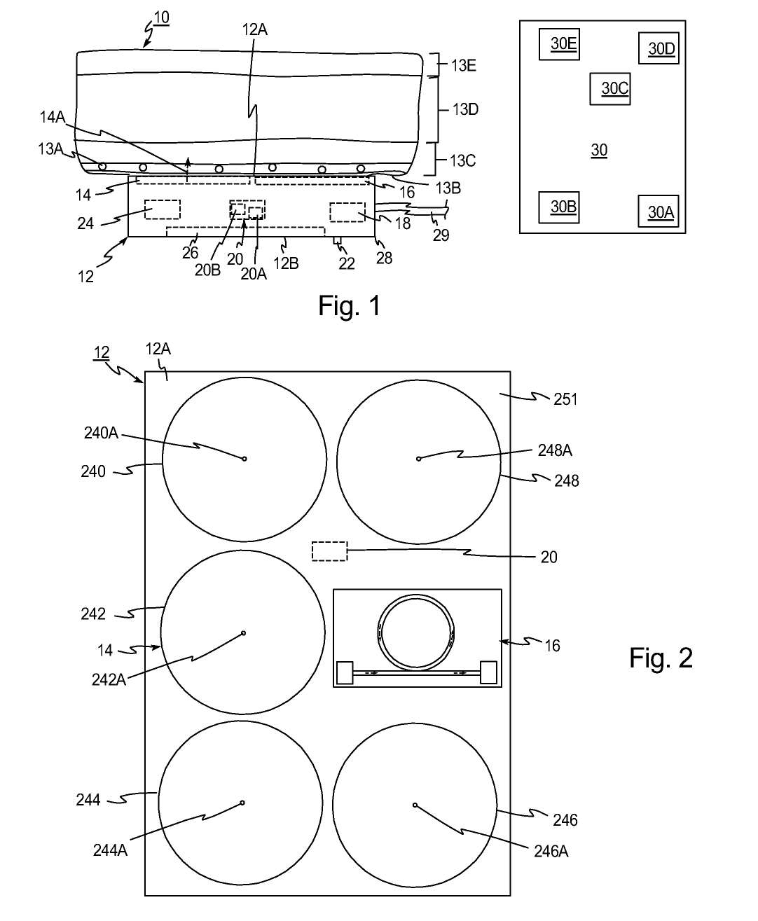

FIG. 1 is a simplified side view of a portion of a living being 10 (illustrated as an amorphous shape) and a body monitor 12. As provided herein, the body monitor 12 can be used to monitor one or more conditions (or analytes) of the being 10. For example, the body monitor 12 can be used to non-invasively monitor the blood glucose level of the being 10. Alternatively, the body monitor 12 can be used to non-invasively monitor one or more other properties of another analyte of the being 10.

As an overview, in certain embodiments, the body monitor 12 is a non-invasive glucose monitor that provides an infrared laser photoacoustic spectroscopy of the subcutaneous tissue, blood, or interstitial fluid 13A (illustrated as small dots for reference) of the being 10 to monitor the glucose. In certain embodiments, the infrared laser may be a quantum cascade laser, or an interband cascade laser, or an infrared diode laser.

As non-exclusive examples, the being 10 can be a human or an animal. In one embodiment, during usage, the body monitor 12 is positioned directly against and in direct contact with the outer skin 13B of the being 10. The epidermis 13C, the dermis 13D, and the hypodermis 13E have also been identified in FIG. 1. As non-exclusive examples, the body monitor 12 can be positioned directly against and in contact with a finger, hand, arm, leg, chest or other region of the body of the human.

The design of the body monitor 12 and the components of the body monitor 12 can be varied to suit the one or more characteristic(s) desired to be monitored. In FIG. 1, the body monitor 12 is a blood glucose monitor and includes (i) a laser assembly 14 (illustrated as a box in phantom), (ii) a detector assembly 16 (illustrated as a box in phantom), (iii) a power source 18 (illustrated as a box in phantom), (iv) a control system 20 (illustrated as a box in phantom), (v) one or more control switches 22 (only one is illustrated in FIG. 1), (vi) a transmitter/receiver 24 (illustrated as a box in phantom), (vii) an output display 26 (illustrated as a box in phantom), and (viii) a monitor housing 28. It should be noted that one or more of these components are optional. For example, the control switches 22 and/or the output display 26 can be replaced by or used in conjunction with an external control system 30 (illustrated as a box) that is used to control the body monitor 12 and display the results of the testing.

In one embodiment, the body monitor 12 monitors the interstitial fluid 13A beneath the skin 13B of the being 10. Interstitial fluid 13A is similar in composition to blood plasma. Further, the glucose levels in the interstitial fluid 13A closely match those in blood. In one embodiment, the body monitor 12 is a portable assembly that is worn or carried by the user. In this embodiment, the body monitor 12 is a wearable glucose monitor based on infrared spectroscopy of the interstitial fluid 13A in being 10.

For example, for a portable assembly, the body monitor 12 can include a wearable feature 29, e.g. a strap, chain, or other means to facilitate wearing of the body monitor 12. As non-exclusive examples, the body monitor 12 can be worn as a watch or pendant.

Alternatively, for example, the body monitor 12 can be a fixed assembly. In this embodiment, the being 10 can approach the body monitor 12 and put their finger, hand, arm, leg, chest or other region of the body against the body monitor 12.

Additionally, or alternatively, the body monitor 12 can be used to directly monitor the blood flowing in the blood vessels of the being 10.

In FIG. 1, the body monitor 12 includes a body side 12A that is positioned directly against the being 10 and an opposed display side 12B.

The laser assembly 14 directs an output beam or collection of output beams 14A at the being 10. In FIG. 1, the laser assembly 14 is positioned adjacent to and directly against the skin 13B of the being 10. In one embodiment, the laser assembly 14 includes one or more compact, surface emitting lasers that generate excitation radiation 14A directed at and through the skin of the being 10. For a glucose monitor, the output beams 14A is used to excite the glucose in the being 10. The laser assembly 14 is described in more detail below in reference to FIG. 2.

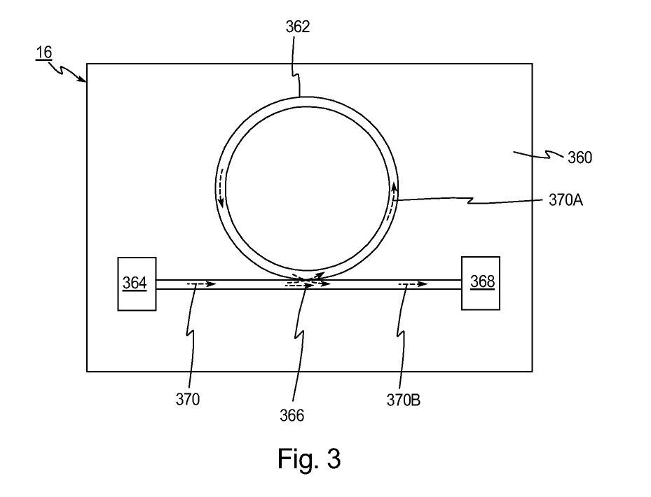

The detector assembly 16 detects the influence of the output beam(s) 14A on the being 10. In FIG. 1, the detector assembly 16 is positioned adjacent to and directly against the skin of the being 10. In one embodiment, the detector assembly 16 is a compact, sensitive photoacoustic detector that monitors photoacoustic waves generated from absorption of the output beams 14A. The detector assembly 16 is described in more detail below in reference to FIGS. 2 and 3.

In certain embodiments, during usage, the laser assembly 14 and the detector assembly 16 are positioned directly against the skin 13B.

The power source 18 provides electrical power to the components of the body monitor 12. For example, for portable applications, the power source 18 can include one or more batteries. The unique design provided herein requires very little power to operate and thus is well suited for battery operation. Alternatively, the power source 18 can be an electrical outlet or another type of power source.

The control system 20 includes one or more processors or embedded processors 20A for controlling the components of the body monitor 12, and is programmed to control the operation of one or more (or all of the) components of the body monitor 12. For example, the control system 20 can control the laser assembly 14 and the detector assembly 16. More specifically, the control system 20 can (i) control the timing of the pulsing of the laser assembly 14 and the wavelengths generated by the laser assembly, and (ii) can receive one or more detector signals from the detector assembly 16 and determine/estimate the condition (e.g. the glucose level) based on the one or more detector signals. Additionally, the control system 20 can include electronic data storage 20B for storing the results from testing.

The one or more control switches 22 can be controlled by the user to control the body monitor 12. Alternatively, the user can control the body monitor 12 using the external control system 30.

The transmitter/receiver 24 can be used to establish a wireless link between the body monitor 12 and the external control system 30. Alternatively, for example, a cord can be used to electrically connect the body monitor 12 and the external control system 30.

The output display 26 displays one or more features of the body monitor 12. Further, the output display 26 can display the results of the tests performed by the body monitor 12 and/or testing options. For example, the output display 26 can include an LED display.

The monitor housing 28 retains the other components of the body monitor 12. For example, the monitor housing 28 can be made of a rigid material. In FIG. 1, the monitor housing 28 is generally rectangular shaped. Alternatively, the monitor housing 28 can have another suitable shape that contours to the area of the living being 10 that is being tested.

As non-exclusive examples, the external control system 30 can be a mobile phone, a tablet, or other embedded or separate computer. Further, the control system 30 can includes one or more processors or embedded processors 30A for controlling the components of the body monitor 12, and one or more electronic data storage devices 30B that store the results. Further, the external control system 30 can include an one or more control switches 30C (only one is illustrated in FIG. 1) (e.g. a keyboard or mouse), an external transmitter/receiver 30D (illustrated as a box in phantom) that communicates with the body monitor 12, and/or an external output display 30E (illustrated as a box in phantom) such as an LED display. It should be noted that external control system 30 can be programmed to perform some or all of the functions of the on-board control system 20.

FIG. 2 is a simplified view of one non-exclusive embodiment of the body side 12A of the body monitor 12 including the laser assembly 14, the detector assembly 16, and the control system 20. The exact wavelength(s) of the output beam or a collection of output beams 14A generated by the laser assembly 14 can be chosen and/or varied according to the properties of the analyte being monitored. For a glucose monitor, the laser assembly 14 can be designed to generate an output beam or a collection of output beams 14A (illustrated in FIG. 1) that are used to excite the glucose in the being 10. For example, Glucose has a strong spectral signature between approximately eight point five (8.5) and ten point five (10.5) microns (μm) that can be used to quantify glucose concentrations. As used herein, the term “Glucose Detection Wavelength Range” shall mean the eight point five (8.5) and ten point five (10.5) microns (μm) wavelength range. In this embodiment, the laser assembly 14 can be designed to generate an output beam or a collection of output beams 14A in the Glucose Detection Wavelength Range.

It should be noted that in certain embodiments, although the glucose signatures are strong in the Glucose Detection Wavelength Range, it can be necessary to acquire data from a range of wavelengths in the Glucose Detection Wavelength Range and/or outside of the Glucose Detection Wavelength Range in order to discriminate the analyte from the other components of interstitial fluid.

In one embodiment, the laser assembly 14 includes a plurality of individual, surface emitting lasers, with each laser generating a separate wavelength in the Glucose Detection Wavelength Range, and each laser being in contact with the skin. With this design, the laser assembly 14 uses a set of fixed wavelength lasers that cover a portion of the Glucose Detection Wavelength Range. Alternatively, one or more of the lasers can generate a separate wavelength the outside the Glucose Detection Wavelength Range for reference.

As one non-exclusive example, the laser assembly 14 can include five separate surface emitting lasers. In this embodiment, the laser assembly 14 includes (i) a first laser source 240 that emits a first output beam 240A (illustrated as a small circle) having a first center wavelength that is in the Glucose Detection Wavelength Range; (ii) a second laser source 242 that emits a second output beam 242A (illustrated as a small circle) having a second center wavelength that is in the Glucose Detection Wavelength Range; (iii) a third laser source 244 that emits a third output beam 244A (illustrated as a small circle) having a third center wavelength that is in the Glucose Detection Wavelength Range; (iv) a fourth laser source 246 that emits a fourth output beam 246A (illustrated as a small circle) having a fourth center wavelength that is in the Glucose Detection Wavelength Range; and (v) a fifth laser source 248 that emits a fifth output beam 248A (illustrated as a small circle) having a fifth center wavelength that is in the Glucose Detection Wavelength Range. In this example, the center wavelength of each output beam is different. Alternatively, one or more of the laser sources could generate an output beam having a center wavelength that is outside the desired detection range (e.g. outside the Glucose Detection Wavelength Range) for background reference.

Alternatively, for another analyte, such as lactose, the laser sources could be designed to generate one or more output beams in a different mid-infrared detection wavelength range.

Further, the laser sources 240-248 can be controlled by the control system 20 to generate a continuous or pulsed beam at different times. For example, the control system 20 can sequentially in time direct (i) a first set of pulses of current to the first laser source 240 to emit a set of first output beams 240A; (ii) a second set of pulses of current to the second laser source 242 to emit a set of second output beams 242A; (iii) a third set of pulses of current to the third laser source 244 to emit a set of the third output beams 244A; (iv) a fourth set of pulses of current to the fourth laser source 246 to emit a set of fourth output beams 246A; and (v) a fifth set of pulses of current to the fifth laser source 248 to emit a set of fifth output beams 248A.

Alternatively, the laser assembly 14 can be designed to include more than five or fewer than five separate surface emitting lasers. For example, the laser assembly 14 can be alternatively designed with two, three, four, or six lasers. In certain embodiments, additional alternative wavelengths may improve the accuracy and/or distribution of the analyte signal.

As provided herein, in certain embodiments, (i) the first output beam 240A directed toward the skin is at least partly absorbed to generate a first photoacoustic signal (or wave) in the epidermis, dermis, or hypodermis; (ii) the second output beam 242A directed toward the skin is at least partly absorbed to generate a second photoacoustic signal in the skin; (iii) the third output beam 244A directed toward the skin is at least partly absorbed to generate a third photoacoustic signal in the epidermis, dermis, or hypodermis; (iv) the fourth output beam 246A directed toward the skin is at least partly absorbed to generate a fourth photoacoustic signal in the epidermis, dermis, or hypodermis; and (v) the fifth output beam 248A directed toward the skin is at least partly absorbed to generate a fifth photoacoustic signal in the epidermis, dermis, or hypodermis. It should be noted that the characteristics of each photoacoustic signal will depend upon the characteristics (e.g. the glucose level) in the interstitial fluid and the wavelength of the output beam.

As provided above, for example, the control system 20 can control each laser 240-246 to pulse at a different time. For example, in one embodiment, the control system 20 can sequentially direct a separate set of pulses of current to (i) the first laser 240 to generate the first output beam 240A that consists of a plurality of sequential first pulses of light having the first center wavelength; (ii) the second laser 242 to generate the second output beam 242A that consists of a plurality of sequential second pulses of light having the second center wavelength; (iii) the third laser 244 to generate the third output beam 244A that consists of a plurality of sequential third pulses of light having the third center wavelength; (iv) the fourth laser 246 to generate the fourth output beam 246A that consists of a plurality of sequential fourth pulses of light having the fourth center wavelength; and (v) the fifth laser 248 to generate the fifth output beam 248A that consists of a plurality of sequential fifth pulses of light having the fifth center wavelength. With this design, each output beam is generated at a different time, such that the timing of the pulses encodes the wavelength and each output beam 240A-248A can be used to acquire information from a different wavelength.

In certain embodiments, with the present invention, the interstitial fluid 13A (illustrated in FIG. 1) is irradiated by modulated output beams 240A-248A. The interstitial fluid 13A absorbs some of the light energy and converts it into a photoacoustic signal which is detected by the photoacoustic detector 16. More specifically, if the wavelength of the output beam 240A-248A coincides with an absorption band of the glucose, the glucose in the interstitial fluid 13A will absorb part of the light. The higher the concentration of the glucose in the interstitial fluid 13A, the more light will be absorbed. The absorbed energy from the light causes local heating and thermal expansion that results in a pressure wave. The more energy absorbed results in larger photoacoustic signals, while less energy absorbed results in smaller photoacoustic signals.

Further, as the timing of the output beam is modulated, the pressure will alternately increase and decrease to generate the photoacoustic signal that is detected by the photoacoustic detector 16. Moreover, different wavelengths will generate different photoacoustic signals.

The center wavelength of each output beam can vary. For a glucose monitor, a non-exclusive list of possible center wavelengths include approximately 940, 990, 1020, 1030, 1080, 1110, 1120, 1150, 1170, and/or 1240 wavenumbers. However, other wavelengths in the mid-infrared range are possible.

It should be noted if it is desired to monitor another analyte, that other center wavelengths can be utilized.

Additionally, it should be noted that the order of firing (pulsing) of the lasers 240-248 can be any arrangement.

Alternatively, the control system 20 can sequentially direct power to the lasers 240-248 in a continuous fashion.

In FIG. 2, five different discrete wavelengths in the Glucose Detection Wavelength Range are used to monitor glucose. Alternatively, the laser assembly 14 can be designed to include more than five or fewer than five separate lasers. Thus, more than five or fewer than five different discrete wavelengths in the Glucose Detection Wavelength Range can be used to monitor the glucose level.

Still alternatively, one or more of the lasers can be built so that its beam is in a mid-infrared range (approximately 2-20 micrometers) but outside the Glucose Detection Wavelength Range. In a non-exclusive example, the photoacoustic signal generated by this laser may provide a background against which to discriminate the glucose-dependent signal.

In FIG. 2, the lasers 240-248 are arranged in two rows, side-by-side in a planar array. Alternatively, the lasers 240-248 can be arranged in another fashion. For example, one or more of the laser sources 240-248 can be arranged in a concentric fashion.

Further, in FIG. 2, each laser 240-248 is a compact, ring shaped, surface emitting, quantum cascade laser, and each laser 240-248 generates a separate laser beam in the MIR frequency range. In one embodiment, each laser 240-248 is less than approximately one millimeter in diameter. In alternative, non-exclusive embodiments, one or more of the lasers 240-248 is less than approximately 0.5, 0.6, 0.7, 0.8, 0.9, 1, 1.1, 1.2, 1.5 or 2 millimeters in diameter. Still alternatively, one or more of the lasers 240-248 can have a different shape or size than described above. In other embodiments, one or more of the lasers could be a vertical cavity surface emitting laser (VCSEL) using interband cascade or diode gain media.

Further, in certain embodiments, each laser 240-248 is not tunable. Moreover, each laser 240-248 can be pulsed to couple with the detector assembly 16. Moreover, in one embodiment, each laser 240-248 should provide sufficient power to be just below the 1 mW/mm2 safety limit.

In one embodiment, the body side 12A of the body monitor 12 includes a substrate 251. Further, one or more of the lasers 240-248 can be grown or built directly on the substrate 251, and/or the detector assembly 16 can be grown or built directly on the substrate 251. Further, the other components (e.g. the control system 20, the control switches 22, and/or the transmitter/receiver 24) of the body monitor 12 can be built or attached directly to the substrate 251 With this design, the body monitor 12 is a compact, durable, monolithic, and integral structure.

Alternatively, one or more of the components can be fixedly secured to the substrate 251.

Still alternatively, one or more of the lasers can be a tunable quantum cascade laser (QCL). With this design, one or more of the tunable lasers can be used to scan or sample the Glucose Detection Wavelength Range or another wavelength range.

In one non-exclusive embodiment, the detector assembly 16 includes a ring shaped photoacoustic detector that detects the photoacoustic signals. In certain embodiments, the photoacoustic detector is relatively small in size and is placed in direct contact with the skin to remove the effects of the air temperature and humidity that occur for a solid to gas photoacoustic measurement. Alternatively, the detector assembly 16 can be designed to include more than one, spaced apart, photo-acoustic detectors, and each photoacoustic detector is in direct contact with the skin.

FIG. 3 is a simplified view of one non-exclusive embodiment of the photo-acoustic detector 16. In this embodiment, the photoacoustic detector 16 is a single micro ring resonator assembly that is a transducer that transduces a photoacoustic wave in the skin into a change in resonance frequency of the micro ring resonator that can be used to estimate the current blood glucose level. Alternatively, the photoacoustic detector 16 can include multiple, micro ring resonator assemblies.

In one embodiment, the micro ring resonator assembly includes a detector substrate 360, a micro ring resonator 362, a probing laser source 364, a waveguide 366, and a beam sensor 368. The design of each of these components can be varied to adjust the characteristics of the photoacoustic detector 16.

In one embodiment, the micro ring resonator 362, the probing laser source 364, the waveguide 366, and/or the beam sensor 368 are grown or formed directly on the rigid detector substrate 360. In this embodiment, the photoacoustic detector 16 is integrated and monolithic. As non-exclusive examples, the detector substrate 360 can be a silicon wafer, fused quartz, InP, (InP/Si), GaAs, or InCaAs. Alternatively, one or more of these components can be fixedly secured to the detector substrate 360.

The micro ring resonator 362 is ring shaped and has a relatively high Q (quality) factor. As a non-exclusive example, the micro ring resonator 362 can have a diameter of less than approximately one millimeter. In alternative, non-exclusive embodiments, the micro ring resonator 362 is less than approximately 0.5, 0.6, 0.7, 0.8, 0.9, 1, 1.1, 1.2, 1.5 or 2 millimeters in diameter. Still alternatively, the micro ring resonator 362 can have a different shape or size than described above. For example, the micro ring resonator 362 can be designed based on the wavelength of the probing laser source 364, and the characteristics of the pressure wave in the skin.

The probing laser source 364 directs a probing beam 370 down the waveguide 366. In one non-exclusive embodiment, the probing laser source 364 generates the fixed wavelength, pulsing, probing beam 370 that is in the visible or near-visible range. As a non-exclusive example, the probing beam 370 can have wavelength of approximately 760 nanometers. The exact wavelength can be designed to suit the characteristics of the micro ring resonator 362.

The waveguide 366 is positioned adjacent to and against the micro ring resonator 362. As a result thereof, a first portion 370A of the probing beam 370 is split and travels in the micro ring resonator 362, and a second portion 370B continues on in the waveguide 366 to the beam sensor 368. The beam sensor 368 can be a photo-diode or other type of sensor that senses the intensity of the second portion 370B that is used to determine the blood-glucose level or other condition.

In this embodiment, the photoacoustic wave in the skin (e.g. generated by the absorption of glucose in the being) against the micro ring resonator 362 causes a change in coupling between the micro ring resonator 362 and the waveguide 366 that changes how much of the probing light 370 stays in the waveguide 366 and how much is transferred to the micro ring resonator 362. Thus, the photoacoustic wave in the skin (e.g. generated by the absorption of glucose in the being) against the micro ring resonator 362 causes a corresponding change in the size of the first portion 370A and second portion 370B.

The amount and type of change will vary depending upon the design of the detector assembly 16. For example, in one design of the detector assembly 16, (i) the amount of light maintained in the waveguide 366 increases (and the amount of light in the micro ring resonator 362 decreases) as the magnitude of photoacoustic wave increases; and (ii) the amount of light maintained in the waveguide 366 decreases (and the amount of light in the micro ring resonator 362 increases) as the magnitude of the photoacoustic wave decreases. In this design, the size of the second portion 370B decreases as the magnitude of the photoacoustic wave increases, and the size of the second portion 370B increases as the magnitude of the photoacoustic wave decreases. This can be monitored by the beam sensor 368.

Alternatively, in another design of the detector assembly 16, (i) the amount of light maintained in the waveguide 366 increases (and the amount of light in the micro ring resonator 362 decreases) as the magnitude of photoacoustic wave decreases; and (ii) the amount of light maintained in the waveguide 366 decreases (and the amount of light in the micro ring resonator 362 increases) as the magnitude of the photoacoustic wave increases. In this design, the size of the second portion 370B increases as the magnitude of the photoacoustic wave increases, and the size of the second portion 370B decreases as the magnitude of the photoacoustic wave decreases. This can be monitored by the beam sensor 368.

As provided above, if the frequency of the output beam 240A-248A (illustrated in FIG. 2) coincides with an absorption band of the glucose, the glucose will absorb part of the light. The higher the concentration (higher percentage) of the glucose in the being 10, the more light will be absorbed and the larger the photoacoustic response. Thus, with the present invention, at certain wavelengths of the output beam 240A-248A, (i) as the concentrations of the glucose increases, the magnitude of the photoacoustic waves increases; and (ii) as the concentrations of the glucose decreases, the magnitude of the photoacoustic waves decreases. Stated in another fashion, the percentage of glucose will determine the size of the second portion 370B sensed by the sensor 368.

With the present design, the micro ring resonator 362 is very sensitive to high frequencies, and not sensitive to low frequencies. This allows the micro ring resonator 362 to easily separate the photoacoustic waves from background noise in the environment.

Further, the time resolution of the micro ring resonator 362 is very short (e.g. in the microsecond regime). Thus, using a fast clock, a time delay between the pulsing of the laser assembly 14 and the detection of the corresponding photoacoustic wave by the micro ring resonator 362 can be monitored. As provided above, the photoacoustic wave is caused by absorption of the beam 14A. Thus, as provided herein, the time delay increases as the depth in the being 10 at which the absorption occurs increases, and the time delay decreases as the depth in the being at which the absorption occurs decreases.

As a result thereof, the time delay (in microseconds) can be converted into depth in tenths of millimeters in the being 10. Thus, in certain embodiments, the time delay (or “echo time”) between the transmitted laser pulse and the received photoacoustic signal can be used to determine a depth of the sampling of the glucose in the being 10. In summary, the time delay between the generation of the output beam 240A-248A and the production of the photoacoustic signal, and the speed of travel of the photoacoustic waves can be used to estimate the depth of the sampling (e.g. the layer of skin in which the absorption occurs). For another example, if the laser light is modulated rather than pulsed, the phase delay between laser amplitude and received signal amplitude may be similarly exploited.

As provided herein, with the present invention, the firing of the laser assembly 14 and the detection with the micro ring resonator 362 can occur very fast, e.g. many times each second. Thus, as non-exclusive embodiments, the present invention allows for the monitoring of the blood glucose level at less than a second, one second, minute, hourly, or daily intervals.

Moreover, with this design, the present invention allows for the monitoring of how and when the blood glucose level is influenced by different foods. For example, the present invention can indicate when a sugary treat causes a spike in the blood glucose level.

Further, because, the firing of the laser assembly 14 and the detection with the micro ring resonator 362 can occur very fast, the present invention can be turned off in between the desired testing intervals. This allows for a portable and battery powered operation.

In certain embodiments, the present invention is directed to a wearable glucose monitor based on quantum cascade lasers and photoacoustic spectroscopy. The ring shaped, quantum cascade lasers, coupled with one or more small, surface contact photoacoustic detector(s) could realize clinically relevant glucose measurements.

As provided above, in certain embodiments, with the present invention, the interstitial fluid 13A is interrogated by pulsed or modulated output beams 240A-248A. The interstitial fluid 13A absorbs some of the light energy and converts it into a photoacoustic signal which is detected by one or more photoacoustic detectors 16. More specifically, if the frequency of the output beam 240A-248A coincides with an absorption band of the glucose, the glucose in the interstitial fluid 13A will absorb part of the light. The higher the concentration of the glucose in the interstitial fluid 13A, the more light will be absorbed. Thus, the photoacoustic signal will increase as the concentration of glucose increases, and the photoacoustic signal will decrease as the concentration of glucose decreases.

It should be noted that, in certain embodiments, the body monitor 12 can be calibrated using existing glucose testing methods. For example, if the body monitor 12 is used as a non-invasive blood glucose monitor, the body monitor 12 can be calibrated for each person using the existing fingerstick method.

In certain embodiments, the present invention allows for the continuous or frequent monitoring of blood glucose levels. This allows for the monitoring of how the blood glucose level of a person is influenced by food, exercise, insulin, or other factors. This information can allow for improved insulin dosing. In certain embodiments, the present invention may be used in conjunction with e.g., an insulin pump to effect a closed-loop control of glucose concentration in the being.

While the particular assembly as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A body monitor for monitoring a condition of a living being, the body monitor comprising:
a monitor housing having a body side that is adapted to be positioned adjacent to the living being;
a first laser source that directs a first output beam at the living being to generate a first photoacoustic signal, the first output beam having a first center wavelength in a mid-infrared range, the first laser source being secured to the monitor housing;
a second laser source that directs a second output beam at the living being to generate a second photoacoustic signal, the second output beam having a second center wavelength in the mid-infrared range, the second center wavelength being different from the first center wavelength, the second laser source being secured to the monitor housing; and
a photoacoustic detector secured to the monitor housing adjacent to the body side, the photoacoustic detector detecting the first photoacoustic signal and the second photoacoustic signal to monitor the condition of the living being, wherein the photoacoustic detector includes a micro ring resonator.

2. The body monitor of claim 1 wherein each laser source includes a surface emitting, ring shaped quantum cascade gain medium that is adapted to be positioned against the living being.

3. The body monitor of claim 1 wherein the micro ring resonator that is adapted to be positioned against the living being.

4. The body monitor of claim 1 wherein the photoacoustic detector including a waveguide that is coupled to the micro ring resonator, and a probing laser source that directs a probing beam down the waveguide.

5. The body monitor of claim 1 wherein the photoacoustic detector monitors a glucose level in the living being.

6. The body monitor of claim 1 further comprising a control system that includes a processor, wherein the control system analyzes a time delay in the first photoacoustic signal to estimate a depth of the sampling.

7. A body monitor for monitoring a condition of a living being, the body monitor comprising:
a monitor housing having a body side that is adapted to be positioned adjacent to the living being;
a laser assembly secured to the monitor housing that (i) directs a first output beam at the living being to generate a first photoacoustic signal, the first output beam having a first center wavelength in a mid-infrared range; and
(ii) directs a second output beam at the living being to generate a second photoacoustic signal, the second output beam having a second center wavelength in the mid-infrared range, the second center wavelength being different from the first center wavelength; and
a photoacoustic detector secured to the monitor housing adjacent to the body side, the photoacoustic detector detecting the first photoacoustic signal and the second photoacoustic signal to monitor the condition of the living being, wherein the photoacoustic detector includes a micro ring resonator.

8. The body monitor of claim 7 wherein the laser assembly includes a surface emitting, ring shaped quantum cascade gain medium that is adapted to be positioned against the living being.

9. The body monitor of claim 7 wherein the photoacoustic detector is a micro ring resonator is adapted to be positioned against the living being.

10. The body monitor of claim 9 wherein the photoacoustic detector includes a waveguide that is coupled to the micro ring resonator, and a probing laser source that directs a probing beam down the waveguide.

11. The body monitor of claim 7 wherein the photoacoustic detector monitors a glucose level in the living being.

12. The body monitor of claim 7 further comprising a control system that includes a processor, wherein the control system analyzes a time delay in the first photoacoustic signal to estimate a depth of the sampling.

13. A method for monitoring a condition of a living being, the method comprising:
directing a first output beam at the living being with a first laser source to generate a first photoacoustic signal, the first output beam having a first center wavelength in a mid-infrared range, the first laser source being secured to the monitor housing;
directing a second output beam at the living being with a second laser to generate a second photoacoustic signal, the second output beam having a second center wavelength in the mid-infrared range, the second center wavelength being different from the first center wavelength, the second laser source being secured to the monitor housing; and
detecting the first photoacoustic signal and the second photoacoustic signal with a photoacousitc detector to monitor the condition of the living being, wherein the photoacoustic detector includes a micro ring resonator.

14. The method of claim 13 wherein the step of directing a first output beam include the first laser source being a surface emitting, ring shaped quantum cascade gain medium that is positioned against the living being.

15. The method of claim 13 wherein the step of detecting includes the a micro ring resonator being positioned against the living being.

16. A body monitor for monitoring a condition of a living being, the living being including an interstitial fluid, the body monitor comprising:
a monitor housing having a body side that is adapted to be positioned against the living being;
a first laser source that directs a first output beam at the interstitial fluid to generate a first photoacoustic signal, the first output beam having a first center wavelength in a mid-infrared range, the first laser source being secured to the monitor housing;
a second laser source that directs a second output beam at the interstitial fluid to generate a second photoacoustic signal, the second output beam having a second center wavelength in the mid-infrared range, the second center wavelength being different from the first center wavelength, the second laser source being secured to the monitor housing;
a third laser source that directs a third output beam at the interstitial fluid to generate a third photoacoustic signal, the third output beam having a third center wavelength in the mid-infrared range, the third center wavelength being different from the first center wavelength and the second center wavelength, the third laser source being secured to the monitor housing; and
a photoacoustic detector secured to the monitor housing adjacent to the body side, the photoacoustic detector detecting the first photoacoustic signal, the second photoacoustic signal and the third photoacoustic signal to monitor the condition of the living being, the photoacoustic detector including a micro ring resonator;
wherein the first laser source, the second laser source, the third laser source, and the micro ring resonator are secured to the mounting housing in a fashion that allows the first laser source, the second laser source, the third laser source and the micro ring resonator to simultaneously be in contact with the living being.

17. The body monitor of claim 16 wherein each laser source includes a surface emitting, ring shaped quantum cascade gain medium.

18. The body monitor of claim 17 wherein the photoacoustic detector including a waveguide that is coupled to the micro ring resonator, and a probing laser source that directs a probing beam down the waveguide.

19. The body monitor of claim 16 wherein the monitor housing has a body side that includes a substrate, and wherein the each of the lasers are positioned on the substrate.

20. The body monitor of claim 16 further comprising a fourth laser source that directs a fourth output beam at the interstitial fluid to generate a fourth photoacoustic signal, the fourth output beam having a fourth center wavelength in the mid-infrared range, the fourth center wavelength being different from the first center wavelength, the second center wavelength, and the third center wavelength.
